(19) United States
(12) Patent Application Publication
(10) Pub. No.: US 2022/0331131 A1
(43) Pub. Date: Oct. 20, 2022

(54) DEVICE AND METHOD FOR CONTROLLING THE FLOW OF EMBOLIC MATERIAL

(71) Applicant: INVATIN TECHNOLOGIES LTD., Katzrin (IL)

(72) Inventors: Elon RESHEF, Haifa (IL); Udi NUSSINOVITCH, Petach Tikva (IL)

(73) Assignee: INVATIN TECHNOLOGIES LTD., Katzrin (IL)

(21) Appl. No.: 17/641,709

(22) PCT Filed: Sep. 10, 2020
(86) PCT No.: PCT/IL2020/050996

§ 371 Date: Mar. 9, 2022

Related U.S. Application Data
(60) Provisional application No. 62/898,575, filed on Sep. 11, 2019.

(51) Int. Cl. A61F 2/848; A61F 2/90; A61F 2/01; A61F 2/92

(52) U.S. Cl. CPC A61F 2/848; A61F 2/90; A61F 2/01; A61F 2/92; A61F 2250/0063

(57) ABSTRACT

A device comprising: a tubular anchoring element configured and dimensioned for implanting about a multifurcation zone of a body lumen dividing a main vessel into at least two branches, to anchor the device therein; a removable sleeve defined by a tubular sidewall, the removable sleeve e being dimensioned for being removably received within a lumen of the anchoring element and extending therein at least the multifurcation zone, wherein the removable sleeve is self-expandable from a radially compressed state defining a delivery configuration into the anchoring element, to a radially expanded state defining a fully expanded configuration, wherein at least a portion of the sidewall, the portion encompassing at least inlets of the at least two branches, comprises a mesh having a mesh size sufficient to allow passage of blood and to deflect the flow of embolic material exceeding a predetermined size.

### CROSS-REFERENCE TO RELATED APPLICATIONS

This application claims priority to U.S. Provisional Patent Application No. 62/898,575 filed on Sep. 11, 2019, the entire contents of which are hereby incorporated by reference herein.

### BACKGROUND

Cardiogenic emboli predispose patients to a variety of pathologies, including stroke or heart and peripheral artery disease. Cardiogenic emboli can arise from a variety of pathologies including Atrial fibrillation (AFib), Left ventricular (LV) hypokinesia and valvular disease, among others.

Cardiogenic emboli account for approximately 20% of strokes each year and are the fifth most prevalent cause of death and a leading cause of disability in the United States.

The term “stroke” is used to describe reduction in the blood supply to the brain or specific areas of the brain caused by blockage of an artery, typically the middle cerebral artery or its branches. Reduction in blood supply can temporarily or permanently impair brain function with the patient experiencing a loss of sight, speech or control of limbs.

Although some success has been achieved with filtering devices, there remains a need for alternative devices and methods for preventing stroke that are safe and can be easily positioned within the lumen of the aortic arch.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a device comprising: a tubular anchoring element configured and dimensioned for implanting about a multifurcation zone of a body lumen dividing a main vessel into at least two branches, to anchor the device therein; a removable sleeve defined by a tubular sidewall, the removable sleeve e being dimensioned for being removably received within a lumen of the anchoring element and extending therein at least the multifurcation zone, wherein the removable sleeve is self-expandable from a radially compressed state defining a delivery configuration into the anchoring element, to a radially expanded state defining a fully expanded configuration, wherein at least a portion of the sidewall, the portion encompassing at least inlets of the at least two branches, comprises a mesh having a mesh size sufficient to allow passage of blood and to deflect the flow of embolic material exceeding a predetermined size.

There is also provided, in an embodiment, a method comprising: implanting a tubular anchoring element about a multifurcation zone of a body lumen dividing a main vessel into at least two branches; delivering, in a radially compressed state, into a lumen of the anchoring element a self-expandable removable sleeve defined by a tubular sidewall, wherein the removable sleeve is self-expandable from the radially compressed state to a radially expanded state defining a fully expanded configuration; and deploying the removable sleeve within the lumen of the anchoring element, by causing the removable sleeve to expand into the expanded state, such that the removable sleeve extends at least the multifurcation zone within the lumen of the anchoring element, wherein at least a portion of the sidewall encompassing at least inlets of the at least two branches, comprises a mesh having a mesh size sufficient to allow passage of blood and to deflect the flow of embolic material exceeding a predetermined size.

In some embodiments, the method further comprises removing the removable sleeve from the lumen of the anchoring element in situ.

In some embodiments, the anchoring element is formed of a self-expandable braided framework able to expand from a radially compressed state in a delivery configuration to a radially expanded state.

In some embodiments, the anchoring member comprises two or more circumferential strut sections disposed at spaced-apart axial positions along a longitudinal dimension thereof.

In some embodiments, each of the two or more circumferential strut sections is a radially expandable stent section configured to engage radially the walls of the body lumen.

In some embodiments, the two or more circumferential strut members are interconnected.

In some embodiments, the anchoring member comprises a fabric sheet attached to and covering circumferentially at least a longitudinal portion of the anchoring element.

In some embodiments, the anchoring member comprises an aperture about an outer wall region encompassing at least the inlets of the at least two branches, wherein the aperture is devoid of any strut members.

In some embodiments, the aperture is defined by a perimeter ring.

In some embodiments, the removable sleeve is configured for in-situ (i) deployment within the lumen of the anchoring element, and (ii) removal from the lumen of the anchoring element.

In some embodiments, the removable sleeve is dimensioned for being fully removably received along its length within the lumen of the anchoring element.

In some embodiments, the removable sleeve is dimensioned for being removably received within the lumen of the anchoring element with respect to a distal section of the removable sleeve.

In some embodiments, when in the radially expanded state, the removable sleeve is configured to radially engage an inner wall of the lumen of the anchoring element, and wherein the engaging is sufficient to prevent axial displacement of the removable sleeve relative to the anchoring element.

In some embodiments, the removable sleeve has a diameter in its expanded state which is greater than a diameter of the lumen of the anchoring element by between 5-15%.

In some embodiments, the removable sleeve comprises one or more interlocking means configured for engaging corresponding features of the anchoring element, for detachably interlocking at least a distal end of the removable sleeve to the anchoring element.

In some embodiments, the interlocking means are configured to prevent axial displacement of the removable sleeve relative to the anchoring element in the proximal direction. In some embodiments, the method further comprises engaging the interlocking means with the corresponding features.

In some embodiments, the removable sleeve comprises at least one radially expandable stent section configured for anchoring at least a proximal end of the removable sleeve to the body lumen. In some embodiments, the method further comprises deploying the at least one radially expandable stent section within the body lumen at a site proximally to a proximal end of the anchoring element.

There is further provided, in an embodiment, a device comprising: a tubular anchoring element configured and dimensioned for implanting about a multifurcation zone of a body lumen dividing a main vessel into at least two branches, to anchor the device therein, wherein the anchoring element comprises an aperture about an outer wall region thereof encompassing at least inlets of the at least two branches, and wherein the aperture is defined by a perimeter ring; a removable insert comprising a resiliently bendable frame having a periphery, and a filter unit within the periphery, the filter unit having a mesh size sufficient to allow passage of blood and to deflect the flow of embolic material exceeding a predetermined size, wherein the removable insert is dimensioned for being received within the ring, and detachably secured thereto using one or more retaining means configured for engaging corresponding features in the ring.

There is further provided, in an embodiment, a method comprising: implanting a tubular anchoring element about a multifurcation zone of a body lumen dividing a main vessel into at least two branches, wherein the anchoring element comprises an aperture about an outer wall region thereof encompassing at least inlets of the at least two branches, and wherein the aperture is defined by a perimeter ring; delivering into a lumen of the anchoring element, a removable insert comprising a resiliently bendable frame having a periphery, and a filter unit within the periphery, the filter unit having a mesh size sufficient to allow passage of blood and to deflect the flow of embolic material exceeding a predetermined size, wherein the removable insert is dimensioned for being received within the ring, and detachably secured thereto using distal and proximal retaining means configured for engaging corresponding distal and proximal features in the ring; engaging the distal corresponding features using the distal retaining means; advancing the removable insert distally, so as to cause the removable insert to resiliently bend at least along a longitudinal dimension thereof aligning the proximal retaining means with the proximal corresponding features; and allowing the removable insert to allowed to resiliently return to its original shape, thereby engaging the proximal corresponding features using the proximal retaining means.

In some embodiments, the anchoring element is formed of a self-expandable braided framework able to expand from a radially compressed state in a delivery configuration to a radially expanded state.

In some embodiments, the anchoring member comprises two or more circumferential strut sections disposed at spaced-apart axial positions along a longitudinal dimension thereof.

In some embodiments, each of the two or more circumferential strut sections is a radially expandable stent section configured to engage radially the walls of the body lumen.

In some embodiments, the two or more circumferential strut members are interconnected.

In some embodiments, the anchoring member comprises a fabric sheet attached to and covering circumferentially at least a longitudinal portion of the anchoring element.

In some embodiments, the removable insert is configured for in-situ (i) deployment within the aperture, and (ii) removal from the aperture.

In some embodiments, the removable insert is dimensioned for being freely movable within a lumen of the anchoring element.

In some embodiments, the periphery has a shape selected from the group consisting of: circular, oval, and rectangular.

In some embodiments, the removable insert is resiliently bendable along at least one of: a longitudinal dimension and a transverse dimension.

In some embodiments, the removable insert is substantially planar.

In some embodiments, the removable insert is pre-shaped with a radius of curvature along at least one of: a longitudinal dimension and a transverse dimension.

In some embodiments, the removable insert comprises one or more struts traversing the frame.

In some embodiments, the removable insert comprises the retaining means at least at a distal end and a proximal end thereof.

In addition to the exemplary aspects and embodiments described above, further aspects and embodiments will become apparent by reference to the figures and by study of the following detailed description.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1F illustrate exemplary anchoring element of the present device, according to some embodiments of the present disclosure;

FIGS. 2A-2C illustrate various configurations of a tubular sleeve of the present device, according to some embodiments of the present disclosure;

FIG. 3 illustrates the present device positioned in the aortic arch, according to some embodiments of the present disclosure;

FIGS. 4A-4C show interconnecting means for a tubular sleeve of the present device, according to some embodiments of the present disclosure;

FIGS. 5A-5B illustrate another embodiment of the present device in which the deflection or filtering function is by a filter insert, according to some embodiments of the present disclosure;

FIGS. 6A-6D illustrate steps is a deployment procedure of a filter insert of the present device, according to some embodiments of the present disclosure;

FIGS. 7-9B illustrate various configurations of retaining means which may be used in conjunction with a filter insert of the present device, according to some embodiments of the present disclosure; and

FIG. 10 shows a device of the present disclosure positioned in the aortic arch, while filter insert covers the openings to the cerebral arteries, according to some embodiments of the present disclosure.

### DETAILED DESCRIPTION

The present invention relates to a device and associated method for controlling the flow of embolic material. Embodiments of the present invention relate to a device and method for preventing migration of cardiogenic emboli to the brain.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Known devices for preventing migration of cardiogenic emboli into he cerebral arteries are designed for temporary or even permanent (indwelling) placement in the aortic arch, and function in capturing emboli or diverting emboli away from the opening to the cerebral arteries.

Indwelling devices typically take the form of a stent or stent graft that is positioned in the aortic arch, with the wall of the device covering the openings to the cerebral arteries. The wall or a portion overlying the openings is configured to filter out emboli while allowing blood to pass through into the cerebral arteries.

Over time the wall of such devices can be fouled or get clogged by embolic material which reduces the flow of blood therethrough, resulting in reduced blood supply to the cerebral arteries. In addition, the stent-like design of such devices relies on outward radial force applied by the wall of the device against the wall of the aortic arch. Contact between the wall of the device and the wall of the aortic arch can induce neointimal growth which further reduces blood flow through the wall of the device.

Accordingly, in some embodiments, the present disclosure provides for a device comprising:

A permanent or indwelling anchoring element, e.g., a stent graft, configured for positioning and extending within a bifurcation, multifurcation, or branching zone of a body lumen, to provide anchorage of the present device therein, and

a removable and/or replaceable filtering or deflection portion, configured for in-situ interconnecting with the anchoring element, and comprising a mesh having a mesh size sufficient to allow the flow of blood while controlling passage of blood content, e.g., embolic material, exceeding a specified particle size.

As used herein, the terms “body lumen” or “biological vessel” refer to a conduit carrying a biological fluid inside the body. Examples of body lumens or biological vessels include arteries, veins, capillaries, ureters, urethra, and the like. A “bifurcation zone,” “multifurcation zone,” or “branching zone” refers to a zone of a lumen which divides or branches into two or more branching lumens, e.g., at the aortic arch.

As is further described herein, the present device can be used in preventing migration of cardiogenic emboli into the cerebral arteries. An embodiment of the present device is configured for anchoring within the aortic arch.

A device of the present disclosure provides advantages over known solutions, including, but not limited to:

Easy removal and/or replacement of the filtering portion only, without the need to remove and/or replace there anchoring element, which may be carried out when the filtering portion if fouled or clogged, or a different type of filter than the one initially implanted is required.

The use of separate anchoring and filtering portion, wherein the filtering portion does rely on applying outward radial force against a wall of the body lumen for anchoring and/or positioning, and thus is not prone to neointimal growth, fouling, and clogging.

The containment of the anchoring element and the filter element to a peripheral dimension of the body lumen (in contrast, e.g., with balloon-, net-, or basket-based devices), generally leaves an open passageway within the body lumen, and thus provides the ability to continue to perform other cardiac procedures after implantation of the device, such as coronary intervention, aortic balloon valvuloplasty, and the like.

According to some embodiments, the device of the present invention includes two main components. A first component is an anchoring element that is configured for anchoring within the body lumen (e.g., aortic arch). In some embodiments, the anchoring element comprises any known stent-like configuration.

The anchoring element has an expandable essentially cylindrical shape and generally serves as an anchoring portion. An anchoring element of the present disclosure is a portion of the present device that firmly engages radially the walls of the body lumen, which causes a growth of the wall into the net of the devices, and strongly anchors it to the body lumen, thus preventing its accidental displacement, according to known physiological processes.

In some embodiments, a second element comprising the present device is a deflection or filtering portion configured for attachment to and/or removal from the anchoring element, including in-situ attachment and/or removal. In some embodiments, the deflection or filtering portion extends along a portion of a length dimension of the anchoring element which encompasses at least the bifurcation zone, to control and/or prevent flow of blood content having particle size above a specified size from entering branching lumens.

In some embodiments, the anchoring element and deflection or filtering portion may be assembled extracorporeally or in-situ, e.g., with the anchoring element delivered and anchored first, followed by delivery and anchoring and/or attachment to the anchoring element of the deflection or filtering portion.

In some embodiments, the deflection or filtering portion comprises a tubular sleeve dimensioned for positioning at least partially within the anchoring element, such that at least a portion of the deflection or filtering portion extending at least the area of the bifurcation zone is positioned within, and overlaps along a length dimension of, the anchoring element. In some embodiments, the tubular sleeve is configured for interconnecting and/or associating with the anchoring element, so as to prevent or minimize axial movement or displacement of the tubular sleeve from a desired positioning in relation to the anchoring element.

In some embodiments, the tubular sleeve can be a woven pliable tube or a polymeric tube with circular cutouts for filtering. The deflection or filtering function can be along the entire length and/or circumference of the sleeve, or at discrete regions thereof. In some embodiments, at least a portion of the wall of the sleeve is permeable to the biological fluid (e.g., blood) and impermeable to particles larger than a specified size.

In some embodiments, the tubular sleeve may be interconnected and/or associated with the anchoring element at one or more interconnecting points, using any one or more of interconnecting means, including hooks, loops, or tines for engaging potions of the anchoring element (e.g., engaging struts or a ring thereof). Alternatively, the interconnecting means can include an expandable element that can be expanded within the anchoring element. The expandable element can frictionally engage the anchoring element and it can include tines for additional engagement. In any case, interconnection between the interconnecting means and the anchoring element is reversible, thus allowing replacement of the tubular sleeve without removal of the anchoring element. Embodiments of the anchoring element can also be removable.

In some embodiments, the tubular sleeve couples to the anchoring element internally thereof at an end portion of the tubular sleeve and/or at least one point along the length of the tubular sleeve, so as to extend therein at least a portion of the length of the anchoring element, which portion corresponds to at least the bifurcation zone. In some embodiments, the tubular sleeve is configured for attaching and/or coupling to the anchoring element internally thereof in at least one point.

In some embodiments, in addition to or as an alternative for, the one or more coupling points, the tubular sleeve comprises one or more anchoring portions for anchoring at least one end and/or at least one point of the tubular sleeve within the body lumen externally to the anchoring element. In such embodiments, the tubular sleeve may extend at least in part from one or more ends of the anchoring element.

In some embodiments, in addition to, or as an alternative for, the one or more coupling points and/or one or more anchoring portions, the tubular sleeve is configured to be a radially expandable tubular sleeve configured for radially expanding within the anchoring element so as to exert sufficient force against a peripheral wall of the anchoring element to resist axial displacement of the tubular sleeve within the anchoring element.

According to some embodiments, an anchoring element of the present disclosure, which may be an expandable essentially cylindrical element configured to firmly engage radially the walls of the body lumen, may comprise an aperture extending at least an area of the a bifurcation zone in the body lumen. In such embodiments, a removable and/or replaceable deflection or filtering portion of the present disclosure may be dimensioned to be received within and detachably secured to the aperture. In some embodiments, the filter element includes a filter frame and a mesh, and is removably attachable to the aperture, for removal and/or attachment in situ.

In some embodiments, anchoring element 12 can include a region of unobstructed flow, i.e., defining an outer wall region with minimal or completely devoid of any strut members and/or scaffolding, wherein the region may align with, face, and encompass a bifurcation zone of a body lumen.

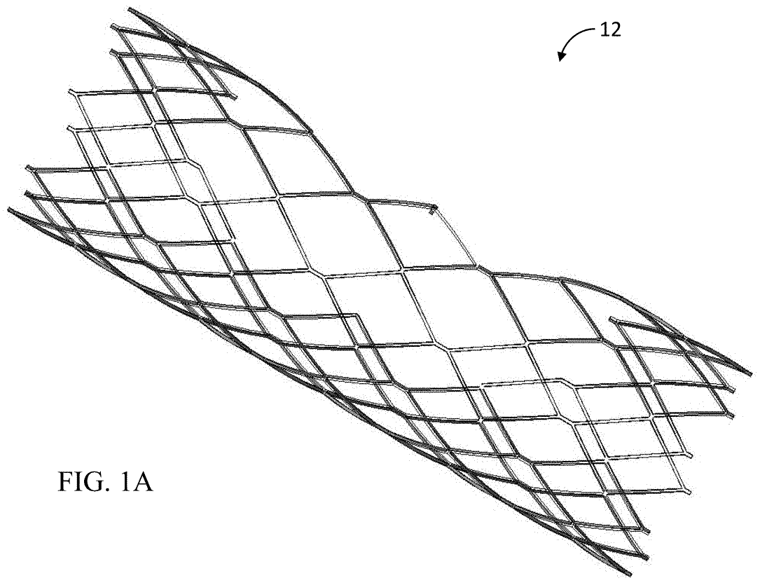

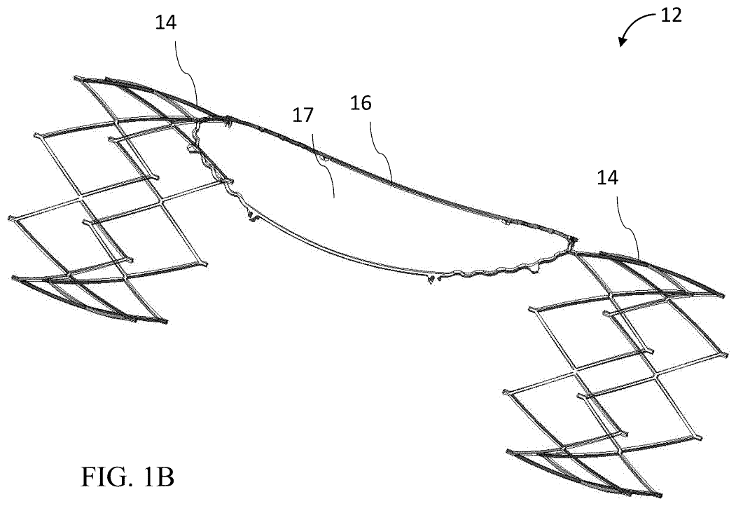

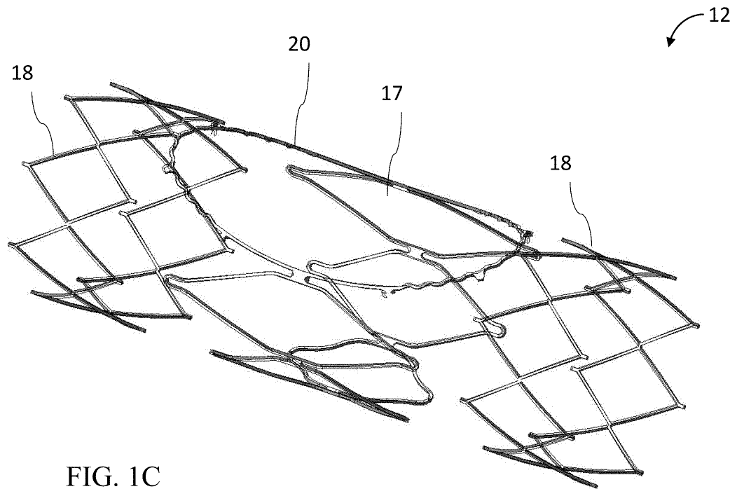

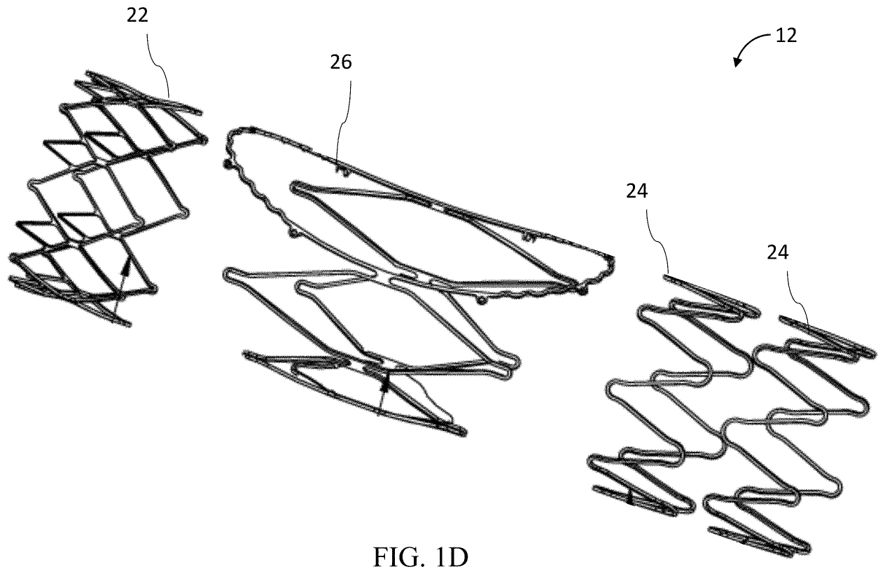

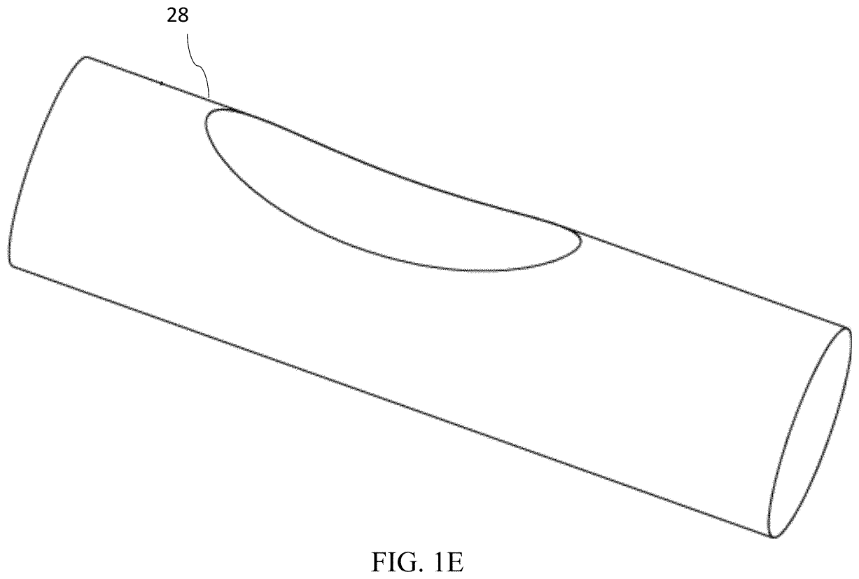

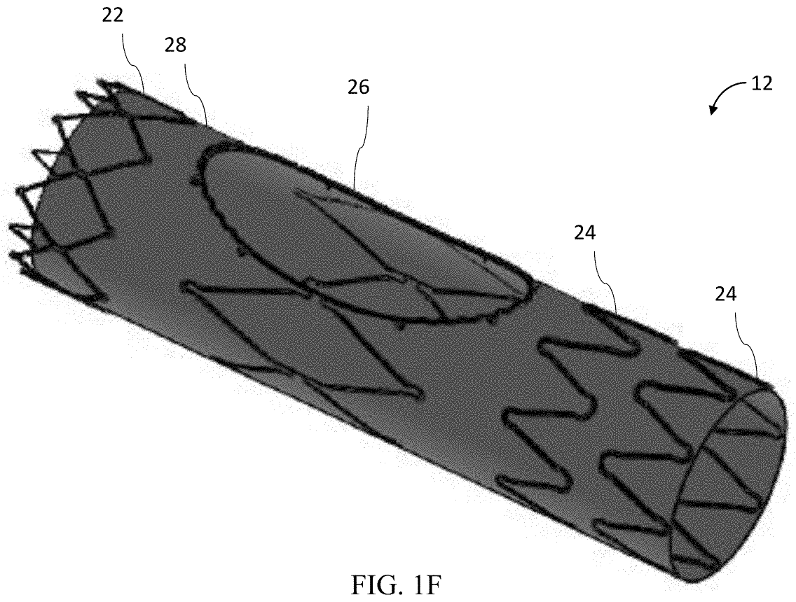

FIGS. 1A-1C illustrate exemplary anchoring elements 12 of the present device, according to some embodiments of the present disclosure.

In some embodiments, anchoring element 12 comprises any suitable stent-like structure or any known stent-like configuration. In some embodiments, anchoring element 12 has an essentially cylindrical shape and generally serves as an anchoring portion. Anchoring element 12 may be configured to firmly engage radially the walls of the body lumen or artery, thus preventing its axial displacement.

In some embodiments, anchoring element 12 comprises a unitary construction, e.g., formed of a self-expandable braided framework able to expand from a radially compressed state in a delivery configuration to a radially expanded state.

In some embodiments, anchoring element 12 may comprise a multi-section construction comprising, e.g., two, three, four, or more connecting members or sections. Each stent section in anchoring element 12 may comprise a circumferential strut member defining, e.g., a waveform, zig-zag, or undulate pattern of struts that can be cut from a Nitinol tube.

With reference to FIGS. 1A-1C, in some embodiments, anchoring element 12 comprises a tubular structure which includes a metal scaffold.

Anchoring element 12 may incorporate a structure that promotes flexibility and conformance to a body lumen, to enable flexure of the anchoring element 12 upon deployment within a body lumen. This flexing structure allows better conformance of the anchoring element to the shape of the body lumen, and exerts less overall pressure against the lumen wall, reducing the potential for trauma.

In FIG. 1A, in some embodiments, anchoring element 12 may comprise a unitary structure defining a tubular metal scaffold.

In FIG. 1B, anchoring element 12 comprises end sections 14 and center section 16. End sections 14 may comprise circumferential strut member defining, e.g., waveform rings or coronas defining expandable stent configured to engage radially the walls of a body lumen. Center section 16 is a hoop section connecting end elements 14 and defining an opening or aperture 17 dimensioned to align with and encompass an area corresponding to at least a bifurcation zone in a body lumen. Sections 14 and 16 may be integrally formed with one another, or attached or coupled to one another to form the structure of anchoring element 12. In some embodiments, the sections of anchoring element 12 can be connected with interlocking links, such as loops or chain-links, that allow the sections to move, but serve to restrict the overall extent of movement. Aperture 17 of anchoring element 12 can be marked via a set of radiological markers to assist alignment.

In FIG. 1C, anchoring element 12 comprises end sections 18 and center section 20. End sections 18 may comprise circular waveform rings or coronas defining expandable stent configured to engage radially the walls of a body lumen. Center section 20 is an expandable stent section configured to engage radially the walls of a body lumen and defining an opening or aperture 17 dimensioned to align with encompass an area corresponding to at least a bifurcation zone in a body lumen. Aperture 17 of anchoring element 12 can be marked via a set of radiological markers to assist alignment.

With reference to FIG. 1D, in some embodiments, anchoring element 12 may comprise a multi-section stent graft incorporating a structure that permits the multiple sections to move relative to another, promoting flexibility and conformance to a body lumen. Movable sections within anchoring element 12 enable flexure of the anchoring element upon deployment within a body lumen.

As can be seen in FIG. 1D, in some embodiments, a multi-section anchoring element 12 of the present disclosure may comprise a plurality of spaced-apart stent sections 22, 24, 26, which may be similar to end sections 14, 16 and/or center sections 18 or 20. In some embodiments, these stent elements may be covered and held together with a fabric or membrane sheet which attaches to and covers circumferentially at least a longitudinal portion of anchoring element 12, forming a housing for the stent sections. This housing can provide an interconnection of the stent sections. FIG. 1E illustrates exemplary covering or membrane, which may be, e.g., a polyethylene terephthalate (PET) or any similar or other suitable material known in the art.

Numerous factors determine the number of stent sections that are affixed to each other comprising anchoring element 12 and the proportion of anchoring element comprising a covering or membrane, including the ability of the sections constrained within a delivery catheter during delivery, and the size of the mesh openings. Generally speaking, a greater proportion of covering or membrane tends to suppress tissue in-growth over the stent. One of ordinary skill would understand how to balance these competing factors, along with other factors, in view of the particular application to determine the ideal number of stents to be utilized.

FIG. 1F shows an exemplary anchoring element 12 comprising stent sections 22, 24, 26, covered in covering 28.

In some embodiments of anchoring element 12, it may be configured for aortic arch placement, wherein anchoring element 12 may be dimensioned as follows:

Between 15-95 mm in length

between 4-8 mm in diameter in a collapsed state for delivery, and

between 10-40 mm in diameter in an expanded deployed state.

Anchoring element 12 can be fabricated from any suitable materials, preferably a material which possesses shape memory properties, e.g., a metal such as stainless steel, nitinol, Elgiloy, other nickel-titanium alloys, or any combination thereof. In some embodiments, sleeve filter frame 31 has a wall thickness within the range of 0.2 mm to 1.2 mm. Alternative configurations of anchoring element 12 can be constructed from a wire mesh or a polymeric tube having cutouts.

The diameter of anchoring element 12 can be constant throughout its length or it can vary such that a diameter of upstream (flow facing) end of anchoring element 12 is larger than a downstream (opposite) end. For example, when anchoring element 12 is in an expanded deployed state, an upstream end can be 20-40 mm in diameter while a downstream end can be 15-35 mm in diameter.

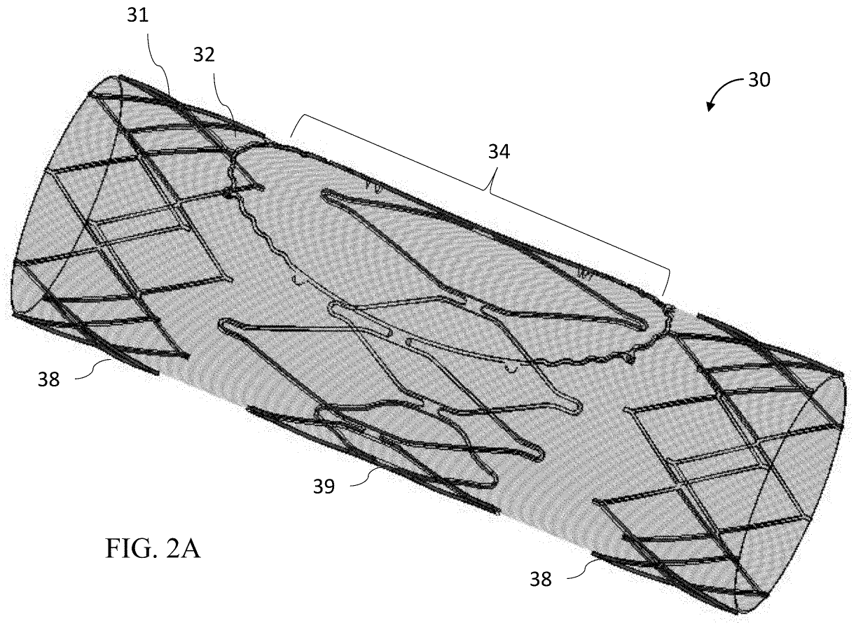

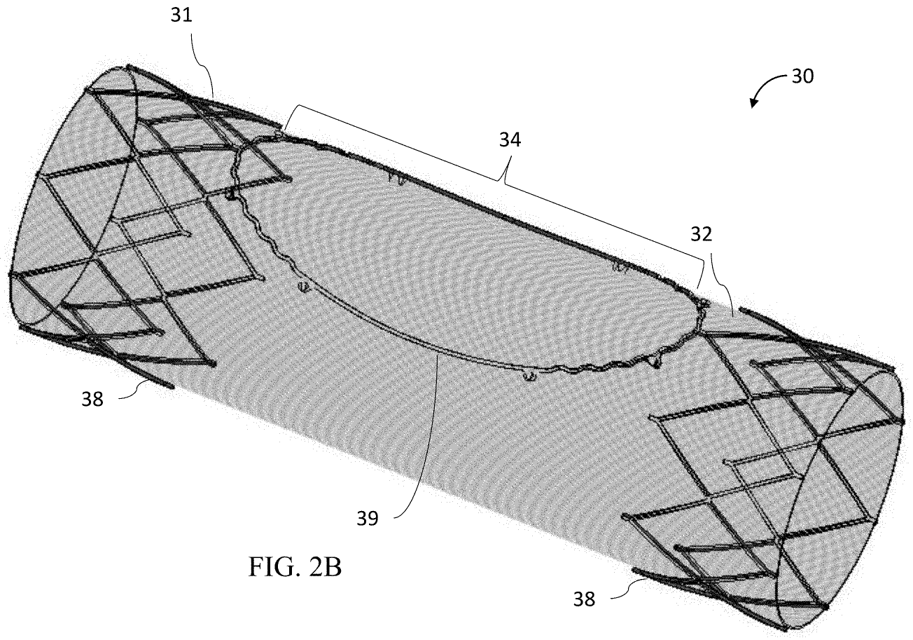

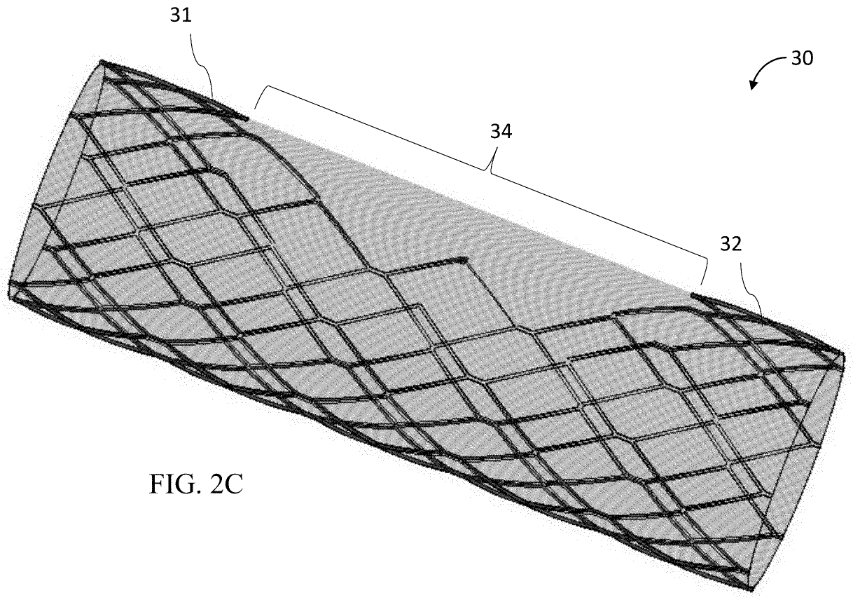

With reference to FIGS. 2A-2C, in some embodiments a device of the present disclosure may comprise a deflection or filtering portion comprising a tubular sleeve 30 dimensioned for positioning at least partially within the anchoring element, such that at least a portion of the deflection or filtering portion encompasses at least the area of the bifurcation zone is positioned within, and overlaps along a length dimension of, the anchoring element. In some embodiments, the tubular sleeve is configured for attachment and/or coupling and/or connecting and/or associating with the anchoring element, so as to prevent or minimize axial movement or displacement of the tubular sleeve from a desired positioning in relation to the anchoring element. In some embodiments, tubular sleeve 30 fits within anchoring element 12 to create a fluid-tight seal. This ensures that a biological fluid flowing through anchoring element 12 flows into sleeve 30.

In some embodiments, tubular sleeve 30 is removably attachable to anchoring element 12. In some embodiments, tubular sleeve 30 is dimensioned to fit inside anchoring element 12, e.g., the diameter of tubular sleeve 30 is smaller than the diameter of the anchoring element. In some embodiments, tubular sleeve 30 is threaded inside the anchoring element.

In some embodiments, tubular sleeve 30 may comprise a region 34 of unobstructed flow, i.e., a region devoid of any scaffolding, wherein said region may align with, face, and encompass a bifurcation zone of a body lumen. Region 34 can be circular oval or rectangular in shape with an area of 5-20 cm². Region 34 can be fabricated by selectively perforating a specific region of solid (polymeric) or tightly meshed tubular sleeve 30 or by selectively weaving the area of region 28 at a lower density. Region 34 can be identified (e.g., outlined) via radiological markers that are visible under, for example, X-ray. Using such markers a physician can position region 34 facing aperture 17 of anchoring element 12, and facing the bifurcation zone when deployed within a body lumen, e.g., the aortic arch.

In some embodiments, tubular sleeve 30 may comprises filter or deflection material, e.g., sleeve filter mesh 32 disposed about at least a portion of a peripheral sidewall of tubular sleeve 30. In some embodiments, sleeve filter mesh 32 covers the entire peripheral sidewall of tubular sleeve 30. In some embodiments, sleeve filter mesh 32 may cover only a portion thereof, e.g., an area corresponding to region 34.

In some embodiments, tubular sleeve 30 comprises a sleeve filter frame such as sleeve filter frame 31. In some embodiments, tubular sleeve 30 comprises a filtering or deflection element, e.g., a sleeve filter mesh such as sleeve filter mesh 32. In some embodiments, sleeve filter mesh 32 is sewn to sleeve filter frame 31. In some embodiments, sleeve filter frame 31 comprises interconnecting means to anchoring element 12. In some embodiments, the interconnecting means can be, e.g., a hook, a loop, a tine, a strut, clips, or any combination thereof. In some embodiments, the interconnecting means is used for interconnecting sleeve filter mesh 32 to tubular sleeve 30 frame 31.

In some embodiments, the diameter of tubular sleeve 30 frame is greater than the diameter of anchoring element 12. In some embodiments, the diameter of tubular sleeve 30 frame is smaller than the diameter of anchoring element 12. In some embodiments, tubular sleeve 30 frame is threaded within anchoring element 12.

In some embodiments, tubular sleeve 30 can be a sleeve-shaped with an overall peripheral sidewall area of between 20-300 cm². In some embodiments, sleeve filter mesh 32 can cover an entire bifurcation zone or region, e.g., the entire area of openings to the cerebral arteries. In some embodiments, tubular sleeve 30 comprises a constant diameter along its length. In some embodiments, the diameter of tubular sleeve 30 gradually increases from end to end.

Tubular sleeve 30 can be elastic or non-elastic and pliable or rigid. A pliable non-elastic tubular sleeve 30 is advantageous in that it can accommodate flow without expanding and without deformation to shape and size of the openings in sleeve filter mesh 32, wherein the openings are configured for allowing biological fluid (e.g., blood) to flow therethrough while preventing flow through of particles such as emboli. The openings in sleeve filter mesh 32 may be a diameter suitable for blocking particles that are larger than 300, 400, 500, 600, 700, 800, 900 or 1000 microns (across the shortest plane of the particle). The openings can be square or circular with an area of 0.4-2.5 mm².

In some embodiments, tubular sleeve 30 can be fabricated from any suitable material. In some embodiments, tubular sleeve 30 can be fabricated from woven fibers with preset fiber thickness, weave density and weave spacing (e.g., woven 20-150 microns wires with hole size of 100-1100 microns) or from a polymeric tube that includes perforations of a preset size, pattern, and density. In some embodiments, openings in a sidewall of tubular sleeve 30 can cover the entire surface, other than region 34 that aligns with, faces and encompasses the bifurcation zone, e.g., the openings to the cerebral arteries.

In some embodiments, sleeve filter frame 31 is formed of any suitable material, preferably a material which possesses shape memory properties, e.g., a metal such as stainless steel, nitinol, Elgiloy, other nickel-titanium alloys, or any combination thereof. In some embodiments, sleeve filter frame 31 has a wall thickness within the range of 0.2 mm to 1.2 mm.

FIGS. 2A-2C illustrate certain embodiments of tubular sleeve 30 frame. In some embodiments, tubular sleeve 30 frame comprises a plurality of support crowns 38, 39. In some embodiments, tubular sleeve 30 frame comprises, e.g., between 2 and 5 support crowns 38 about each end thereof. In some embodiments, tubular sleeve 30 frame comprises a center support crown 39. In some embodiments, tubular sleeve 30 frame comprises two support crowns 38 at both ends thereof and one center support crown 39. In some embodiments, center support crown 39 comprises a hoop section connecting end support crowns 38 and defining an opening or aperture dimensioned to encompass an area corresponding to at least a bifurcation zone in a body lumen.

In some embodiments, tubular sleeve 30 frame comprises a single elongated crown, as can be seen in FIG. 2C.

In some embodiments, sleeve filter mesh 32 is between 30-230 mm in length and 15-45 mm in diameter. In some embodiments, sleeve filter mesh 32 can be fabricated from woven fibers with preset fiber thickness, weave density and weave spacing (e.g., woven 20-150 microns wires with hole sides of 100-1100 microns) or from a polymeric tube that includes perforations of a preset size, pattern, and density. In some embodiments, sleeve filter mesh 32 can be elastic or non-elastic and pliable or rigid. In some embodiments, sleeve filter mesh 32 comprises a wire mesh.

In some embodiments, sleeve filter mesh 32 is configured for allowing biological fluid (e.g., blood) to flow therethrough while preventing flow through of particles such as emboli. In some embodiments, sleeve filter mesh 32 comprises pores with a diameter suitable for blocking particles that are larger than 300, 400, 500, 600, 700, 800, 900 or 1000 microns (across the shortest plane of the particle). In some embodiments, sleeve filter mesh 32 comprises pores which are 50 to 250 microns. In some embodiments, sleeve filter mesh 32 comprises pores which are smaller than 300, 400, 500, 600, 700, 800, 900, or 1000 microns. In some embodiments, a pore may have any shape. In some embodiments, sleeve filter mesh 32 comprises pores having a surface area of less than: 3.0 mm², 2.7 mm², 2.5 mm², 2.4 mm², 2.3 mm², 2.2 mm², 2.1 mm², 2.0 mm², 1.9 mm², 1.8 mm², 1.7 mm², 1.6 mm², 1.5 mm², 1.4 mm², 1.3 mm²,1.2 mm2, 1.1 mm² and 1 mm². In some embodiments, sleeve filter mesh 32 comprises pores having a surface area of 0.3-3.5 mm². In some embodiments, sleeve filter mesh 32 comprises pores having a surface area of 0.4-2.5 mm². In some embodiments, sleeve filter mesh 32 comprises pores having a surface area of 0.8-2.0 mm².

In some embodiments, tubular sleeve 30 is removable and replaceable. In some embodiments, removing or replacing tubular sleeve 30 is performed in-situ without removal of the anchoring element. In some embodiments, removing or replacing tubular sleeve 30 is performed by the delivery catheter.

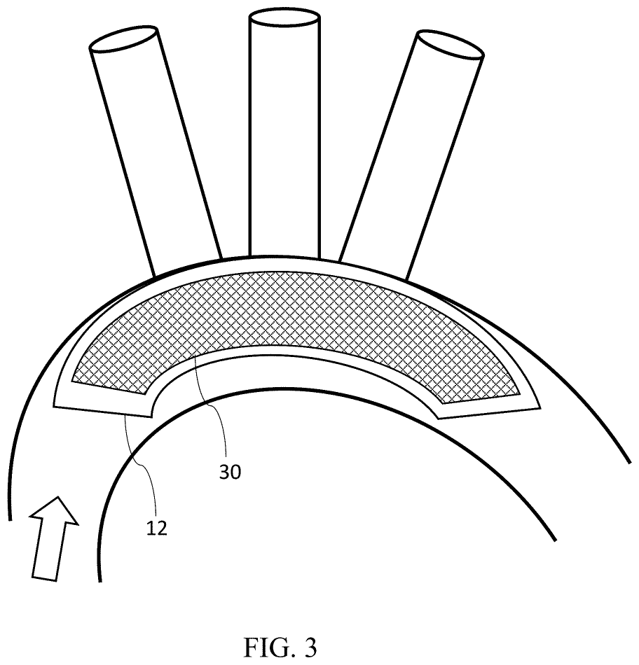

In some embodiments, tubular sleeve 30 is installed inside the anchoring element, before the implanting procedure. In some embodiments, tubular sleeve 30 and the anchoring element implanted as a single unit. In some embodiments, the anchoring element 12 is implanted first, and tubular sleeve 30 may be implanted later as the anchoring element is in position. FIG. 3 illustrates the present device, positioned in the aortic arch, while tubular sleeve 30 covering the openings to the cerebral arteries.

In some embodiments, tubular sleeve 30 may be configured for being at least partially received within a lumen of anchoring element 12, and attached and/or coupled and/or connected and/or associated with anchoring element 12, so as to prevent or minimize axial movement or displacement of tubular sleeve 30 from a desired positioning in relation to the anchoring element.

In some embodiments, tubular sleeve 30 has an essentially cylindrical shape. In some embodiments, tubular sleeve 30 may have a unitary construction, or may comprise a multi-section construction comprising, e.g., two, three, four, or more connecting members or sections. Each section in tubular sleeve 30 may comprise a waveform, zig-zag, or undulate pattern of struts that can be cut from a Nitinol tube or any other suitable material known in the art.

In some embodiments, tubular sleeve 30 may be configured for deployment within anchoring element 12 so as to limit or prevent axial movement of tubular sleeve 30 within anchoring element 12, using one or more associating, interconnecting, and/or anchoring means.

In some embodiments, tubular sleeve 30 is an expandable tubular sleeve 30 which may be configured to be deployed at least partially within anchoring element 12, and be retained in a desired location in relation to anchoring element 12 based, e.g., on applying outward radial force against a wall of anchoring element 12. In such embodiments, tubular sleeve 30 may have a diameter in its expanded deployed state which is slightly greater than an expanded diameter of anchoring element 12, e.g., by 10%, or by between 5-15%.

In some embodiments, anchoring element 12 and tubular sleeve 30 may form a stent-within-a-stent configured. The tubular sleeve 30 may be the same diameter as the anchoring element 12. Alternatively, the tubular sleeve 30 may have a larger diameter than the anchoring element 12 to ensure that tubular sleeve 30 expands tightly against the interior surface of the anchoring element 12. Generally speaking, a tubular sleeve 30 that has the same diameter or a larger diameter than that of the anchoring element 12 will, upon expansion, exert an outwardly directed radial force against the inner surface of the anchoring element 12 that is sufficient in creating and maintaining an adequate fit.

In some embodiments, in addition or alternatively to being an expandable tubular sleeve 30, tubular sleeve 30 may be configured to interconnect to anchoring element 12 at one or more interconnecting points, e.g., at a first or distal end of tubular sleeve 30, via interconnecting means configured to limit or prevent axial movement of tubular sleeve 30 within anchoring element 12.

In some embodiments, in addition or alternatively to being an expandable tubular sleeve 30, and in addition or alternatively to being configured to interconnect to anchoring element 12 at one or more interconnecting points, tubular sleeve 30 may further comprise an expandable anchoring section 52 configured to anchor tubular sleeve 30 to the body lumen at a second end thereof, e.g., a proximal end thereof.

In some embodiments, some or more of these deployment means may be combined in any configuration within tubular sleeve 30, e.g., tubular sleeve 30 may comprise at least an expandable portion, at least one interconnecting means, and/or at least one anchoring section.

In some embodiments, tubular sleeve 30 comprises one or more interconnecting means configured to detachably interconnect or interlock tubular sleeve 30 within anchoring element 12, as tubular sleeve 30 becomes deployed within the lumen of anchoring element 12. In some embodiments, the interconnecting means function as coupling or engagement members to couple/engage tubular sleeve 30 with the anchoring element 12.

In some embodiments, any suitable interconnecting and/or association elements may be employed to interconnect sleeve 30 to anchoring element 12, e.g., hooks, spikes, tines, prongs and/or any other suitable interconnecting means. In some embodiments, any such interconnecting means may be configured to detachably interlock at least one end, e.g., a distal end, of tubular sleeve 30 into anchoring element 12. In some embodiments, such detachable interlocking or interconnecting means may be configured to extend through interstices of anchoring element 12 and thereafter catch on, e.g., struts or any other structural element of anchoring element 12. In some embodiments, detachable interlocking means, e.g., spikes, tines, prongs, and/or barbs may be configured for engaging a covering or membrane of anchoring element 12.

In some embodiments, interconnecting means may be positioned at a distal and/or proximal ends of tubular sleeve 30, and/or at any other various predetermined locations along the length of tubular sleeve 30.

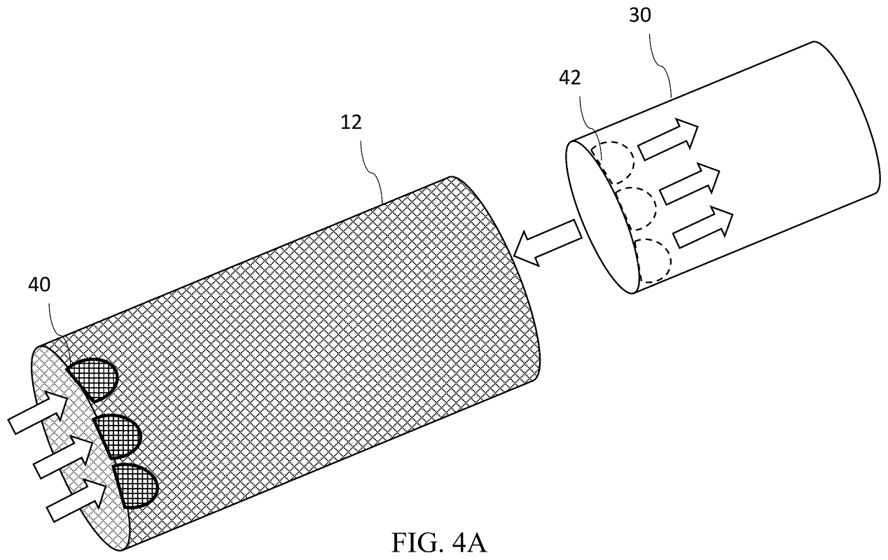

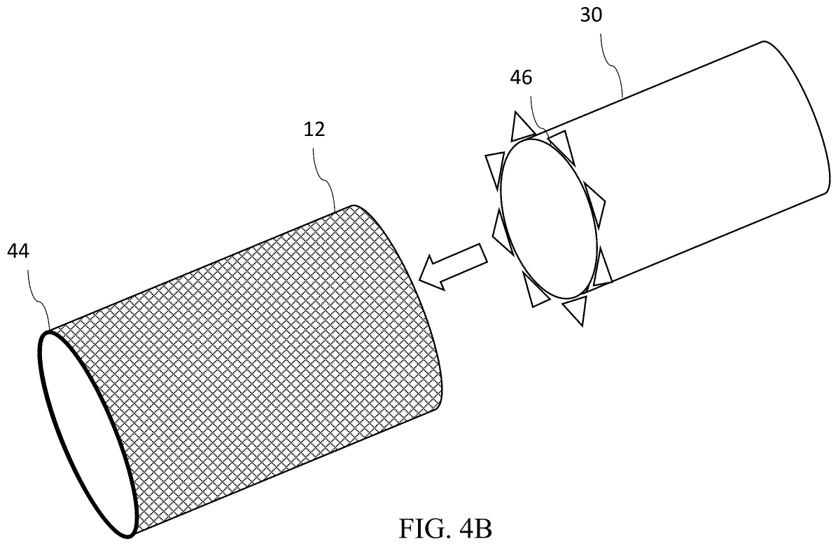

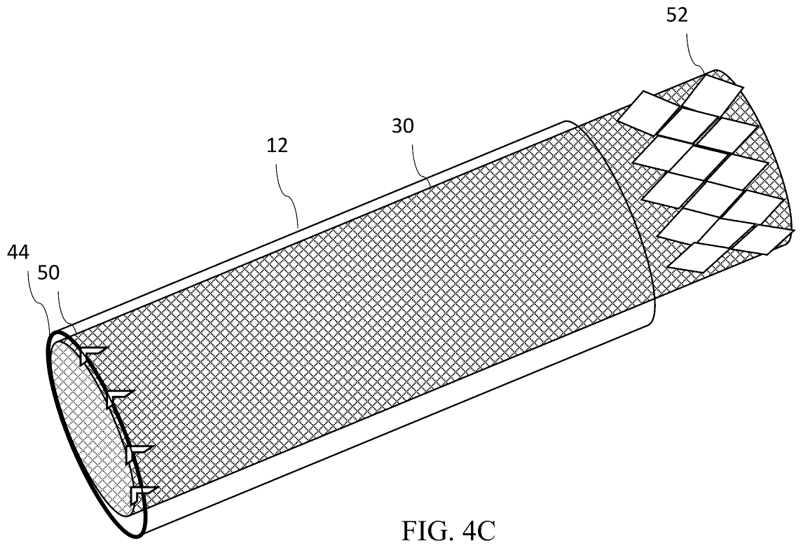

FIGS. 4A-4C illustrate alternative attachment and/or association configurations of tubular sleeve 30 and anchoring element 12.

FIG. 4A shows ‘pockets’ 40 in anchoring element b that are configured to engage, e.g., protrusions or extensions 42 in tubular sleeve 30.

FIG. 4B shows spike ring 44 in anchoring element 12 that engages spikes 46 in tubular sleeve 30.

FIG. 4C shows interconnecting tines 50 configured to engage a distal ring 44 of anchoring element 12, in combination with an anchoring section configured to anchor a proximal end of tubular sleeve 30 at a body lumen, externally of anchoring element 12.

In some embodiments, at least a first end, e.g., a proximal end, of sleeve 30 includes an expandable anchor section 52 that can be stent-like in configuration and can be self or balloon expandable. A second, e.g., distal, end of tubular sleeve 30 can include interconnecting elements for interconnecting tubular sleeve 30 to anchoring element 12.

In some embodiments, tubular sleeve 30 can fit entirely into anchoring element 12 or it can be attached thereto in an overlapping configuration in which attachment elements are attached to anchoring element and anchor section 52 is anchored in the body lumen downstream of anchoring element 12.

Delivery of the present device into the body lumen, e.g., the aortic arch, is carried out using a delivery catheter configured to work with a 0.035″ or 0.038″ guidewire (GW). The catheter includes radio opaque (or other) markers for facilitating accurate and proper tracking, positioning and deployment of the device. The catheter includes a handle to enable proper manipulation through the vasculature, ports for the GW, ports for contrast media injection, mechanisms for device unsheathing or re-sheathing for device repositioning. The handle is connected to an inner tube 60-140 cm in length with a diameter of 28 to 10 Fr. The inner tube is connected to a radio-opaque tip and includes features to enable proper tracking and deployment including markers, stoppers, re-sheathing elements and others. The inner tube is covered by an outer tube/Graft Cover/Introducer Sheath. The device can be pre-mounted on the catheter or is crimped and mounted on the catheter at the interventional laboratory.

The procedure resembles Aortic arch interventional stenting. An introducer sheath is positioned at an access site (trans femoral, trans aortic or trans apical) and the device mounted on the distal end of the catheter is advanced through the introducer sheath to the aortic arch using standard methods of interventional navigation.

The present device is positioned such that tubular sleeve 30 completely covers and encompasses all the branches of the aortic arch that supply the brain and upper body. The catheter and tubular sleeve 30 are designed to enable re-sheathing retrieval or repositioning is needed. When the optimal position is achieved, the device is released from the catheter. The procedure enables accurate and safe deployment and allows for partial release of the anchoring ends of tubular sleeve 30 and/or anchoring element 12, rapid pacing to increase heart rate to over 200 bpm so that the flow is lowered to minimum and the chance that the device will be pushed by stroke of blood is lowered, prevent stent migration during deployment, and stabilization additional devices or others.

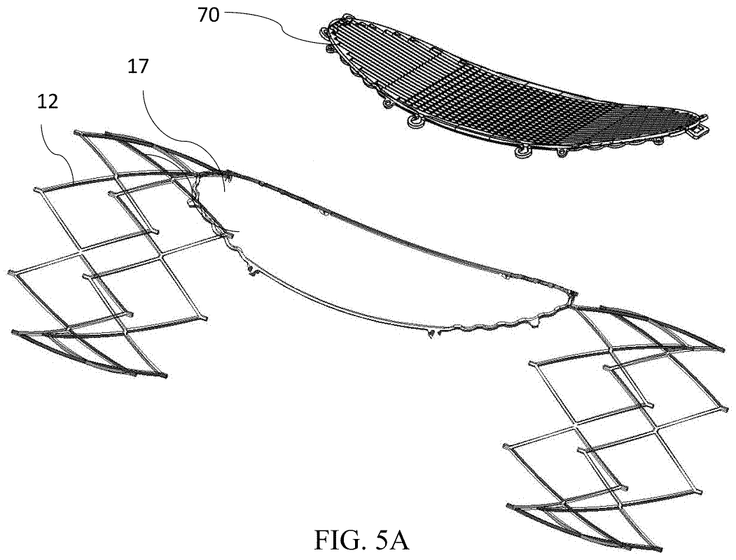

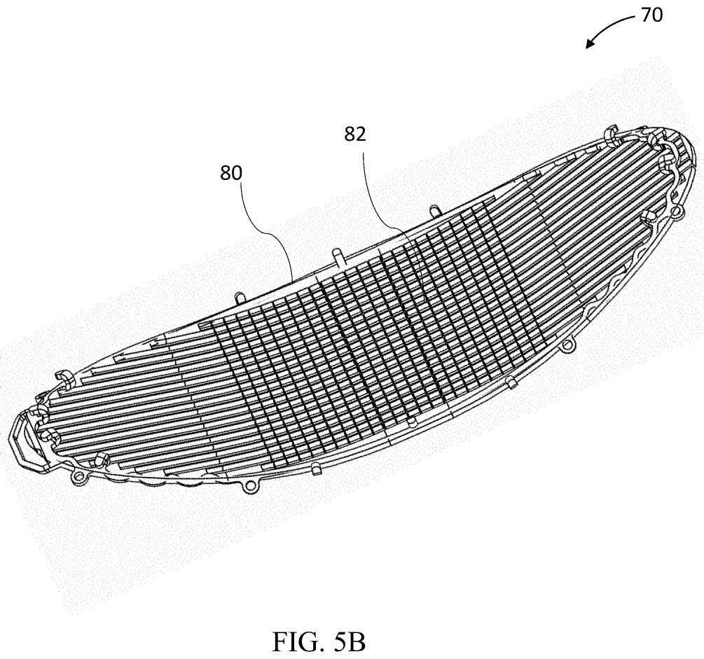

FIGS. 5A-5B illustrate another embodiment of the present device in which the deflection or filtering function is by a filter insert 70. In some embodiments, anchoring element 12 comprises an aperture such as aperture 17 in FIGS. 1B, 1C, defined by a center section of anchoring element 12, such as center sections 16, 20 in FIGS. 1B, 1C. In some embodiments, aperture 17 is defined by a perimeter hoop or ring section configured for receiving filter insert 70. In this disclosure, any references to aperture 17 include the aperture itself and a surrounding ring or hoop defining aperture 17.

In some embodiments, aperture 17 is located within a side wall of the anchoring element. In some embodiments, aperture 17 is positioned so as to encompass a bifurcation zone of a body lumen, when the device of the present disclosure is deployed. In some embodiments, filter insert 70 is configured and dimensioned to be removably tightly received within aperture 17 and detachably secured thereto. In some embodiments, anchoring element 12 has a wall thickness within the range of 0.2 mm to 1.2 mm. In some embodiments, filter insert 70 defines, e.g., a circular, oval, or rectangular in shape, with an area of between 5-20 cm² and dimensioned to be tightly received within aperture 17.

In some embodiments, insert filter 70 has a periphery frame 80 that is a resiliently bendable frame made, e.g., of a metal wire. In some embodiments, filter frame 80 is resiliently bendable along at least one of a longitudinal and a transverse dimensions thereof. In some embodiments, filter frame 80 is made of any suitable material, preferably a material which possesses shape memory properties, e.g., stainless steel, nitinol, Elgiloy, other nickel-titanium alloys, or any combination thereof. In some embodiments, filter insert 70 may be substantially planar or may be pre-shaped with a radius of curvature along at least one of a longitudinal and a transverse dimensions thereof, such that the filter insert 70 may be concave along at least one dimension thereof.

In some embodiments, filter frame 80 comprises one or more longitudinal and/or transverse struts, e.g., in the form of crisscross pattern.

In some embodiments, insert filter 70 further comprises a blood permeable filtering unit, e.g., filter 82, within the periphery for preventing embolic particles from passing therethrough into branching body lumens, e.g., side vessels of the aortic arch. The frame may be elongate, e.g., ovoid.

Depending on the characteristics of filter 82, embolic material may be temporary trapped in the filter 82. The filter 82 may include a filter material. Alternatively, or in addition, the filter 82 may include or be made of a porous material. In any example of the devices of the disclosure, the filter 82 material can include braided, woven, or clustered material. In certain aspects, the filter 82 material can include laminated mesh. For example, the mesh can include polymeric film, e.g., perforated polymeric film. Alternatively, or in addition, the filter 82 may have characteristics that the embolic material glides or slides along a surface thereof, thus deflecting embolic debris past the side branch vessels.

In some embodiments, filter insert 70 comprises a wire mesh, such as filter 82. In some embodiments, filter insert 70 faces the openings to a cerebral artery. In some embodiments, filter insert 70 is positioned such that it completely covers all the branches of the aortic arch that supply the brain and upper body. In some embodiments, filter insert 70 can extend along the entire side wall. In some embodiments, filter insert 70 extends on less than 70%, 60%, 50%, 40%, 30%, or 20% of the entire length of the present device. In some embodiments, filter insert 70 is permeable to a biological fluid (e.g., blood) and impermeable to particles larger than 300 microns. In some embodiments, filter insert 70 is configured for preventing the flow of particles such as emboli into an artery.

In some embodiments, filter insert 70 comprises a flexible metal. In some embodiments, a filter as described herein comprises pores which are 50 to 250 microns. In some embodiments, a filter as described herein comprises pores which are smaller than 300, 400, 500, 600, 700, 800, 900, or 1000 microns. In some embodiments, a pore may have any shape. In some embodiments, a filter as described herein comprises pores having a surface area of less than: 3.0 mm², 2.7 mm², 2.5 mm², 2.4 mm², 2.3 mm², 2.2 mm², 2.1 mm², 2.0 mm², 1.9 mm², 1.8 mm², 1.7 mm², 1.6 mm², 1.5 mm², 1.4 mm2, 1.3 mm², 1.2 mm2, 1.1 mm² and 1 mm².

In some embodiments, a filter unit as described herein comprises pores having a surface area of 0.3-3.5 mm². In some embodiments, a filter as described herein comprises pores having a surface area of 0.4-2.5 mm². In some embodiments, a filter as described herein comprises pores having a surface area of 0.8-2.0 mm².

In some embodiments, filter insert 70 may be removable, insertable, and/or replaceable with respect to anchoring element 12. In some embodiments, filter insert 70 may be in-situ removable, insertable, and/or replaceable with respect to anchoring element 12.

In some embodiments, removing or replacing the filter element is performed in-situ without removal of the anchoring element. In some embodiments, removing or replacing the filter element is performed by the delivery catheter. In some embodiments, the filter insert 70 is installed in the aperture 17, before the implanting procedure. In some embodiments, the filter insert 70 and the anchoring element 12 are implanted as an assembled device. In some embodiments, the anchoring element 12 may be implanted first, and the filter insert 70 may be deployed later, with the anchoring element in position.

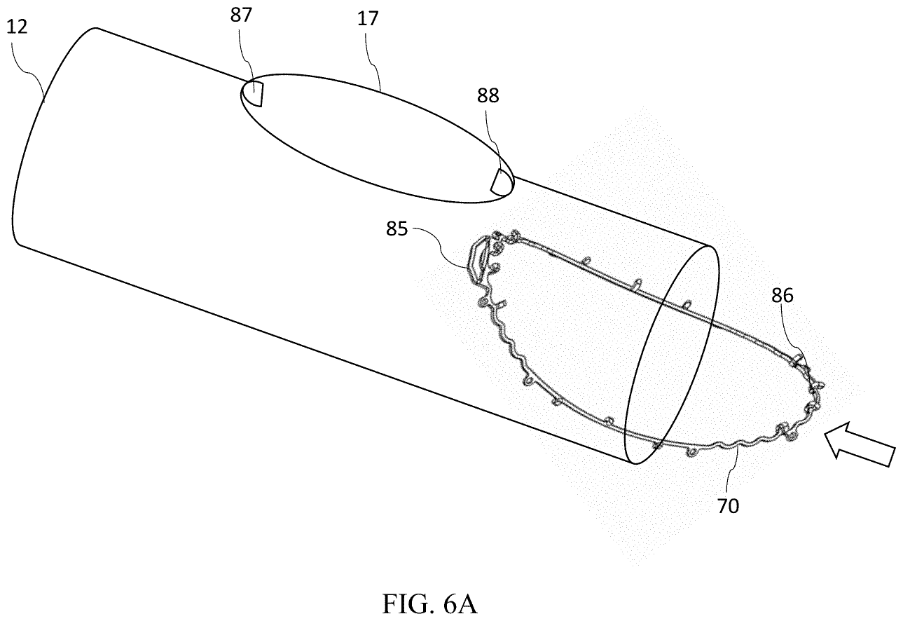

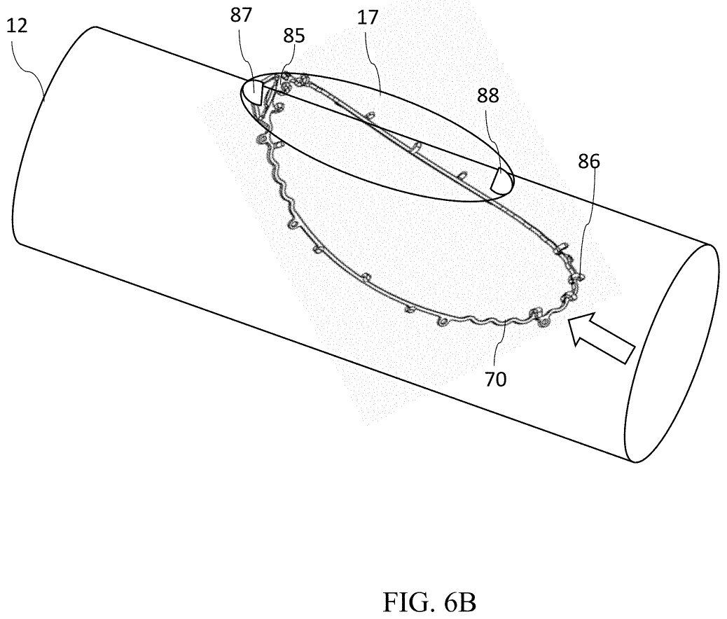

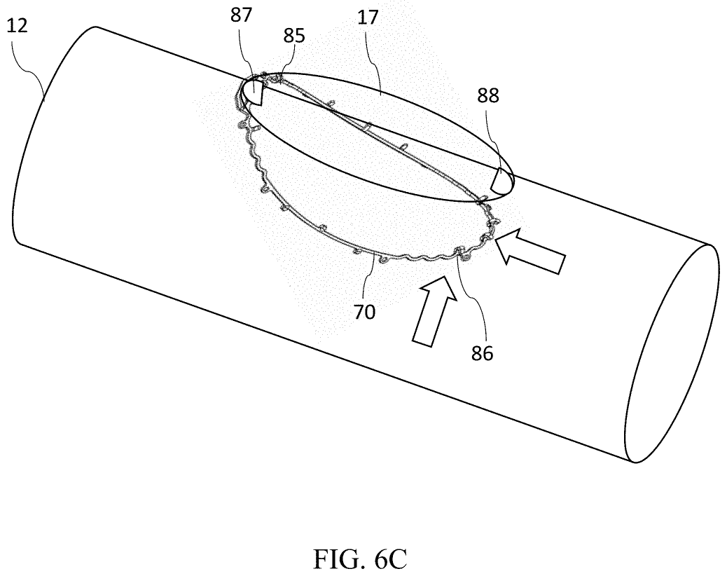

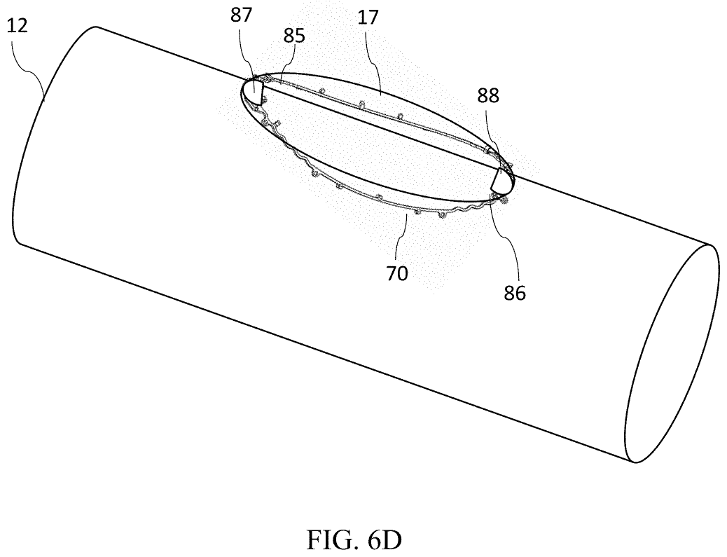

With reference to FIGS. 6A-6D, in some embodiments, during deployment, filter insert 70 may be delivered to a bodily site where anchoring element 12 is deployed. In some embodiments, anchoring element 12 may be deployed such that aperture 17 corresponds to and aligns with an area corresponding to at least a bifurcation zone in a body lumen. In some embodiments, a distal end of filter insert 70 may comprise one or more retaining elements 85 configured to engage a hoop or ring defining aperture 17 within anchoring element 12, and/or one or more corresponding features 87 within the hoop or ring defining aperture 17.

Accordingly, with reference to FIG. 6A, filter insert 17 may be advanced by a suitable delivery means, e.g., a gripper, within anchoring element 12 such, as seen in FIG. 6B, that the distal retaining elements 85 engage the hoop or ring defining aperture 17, and/or the corresponding features 87 within hoop or ring defining aperture 17. With reference to FIG. 6C, upon engagement, filter insert 70 may be further advanced by the delivery means, such that the further advancing causes filter insert 70 to slightly bend or deform along at least one of a longitudinal and a transverse dimensions thereof, wherein the bending or deformation causes at reduction in least one of a longitudinal and a transverse nominal footprint of filter insert 70. Upon such bending and reduction in nominal footprint, retaining means at an opposite end of filter insert 70 may be brought into alignment with an opposite end of the hoop or ring defining aperture 17, and, with reference to FIG. 6D, filter insert 70 may be allowed to resiliently return to its original shape, thus engaging the opposite end of the hoop or ring defining aperture 17, or corresponding feature therein.

In some embodiments, in order to remove a filter insert 70 deployed within an aperture 70 of an anchoring element 12, the order of the operations may be reversed. Accordingly, a delivery tool may be used to grip and advance a proximal end of filter insert 70 so as to slightly bend or deform along at least one of a longitudinal and a transverse dimensions thereof therein disengaging a proximal end of hoop or ring defining aperture 17, or corresponding feature therein. The delivery tool may then be used to lower the proximal end of filter insert 70 towards a center of a lumen of anchoring element 12, and the pull back filter insert 70 so as to disengage a distal end of hoop or ring defining aperture 17, or corresponding feature therein.

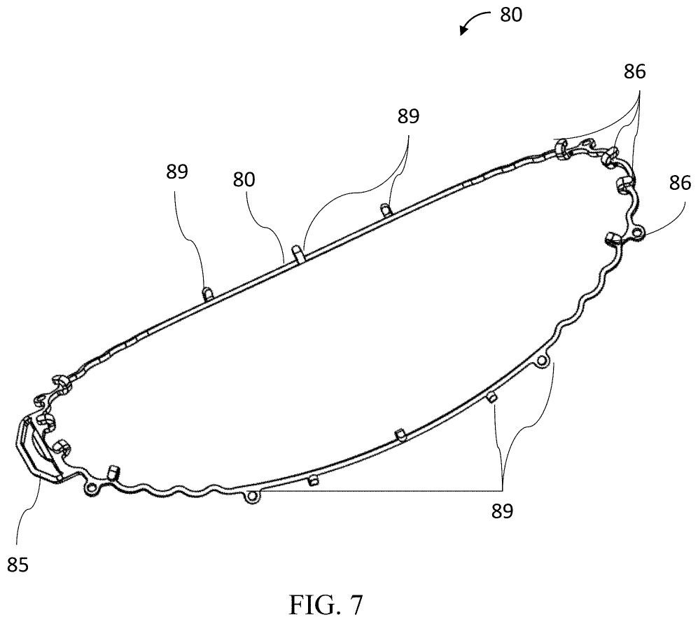

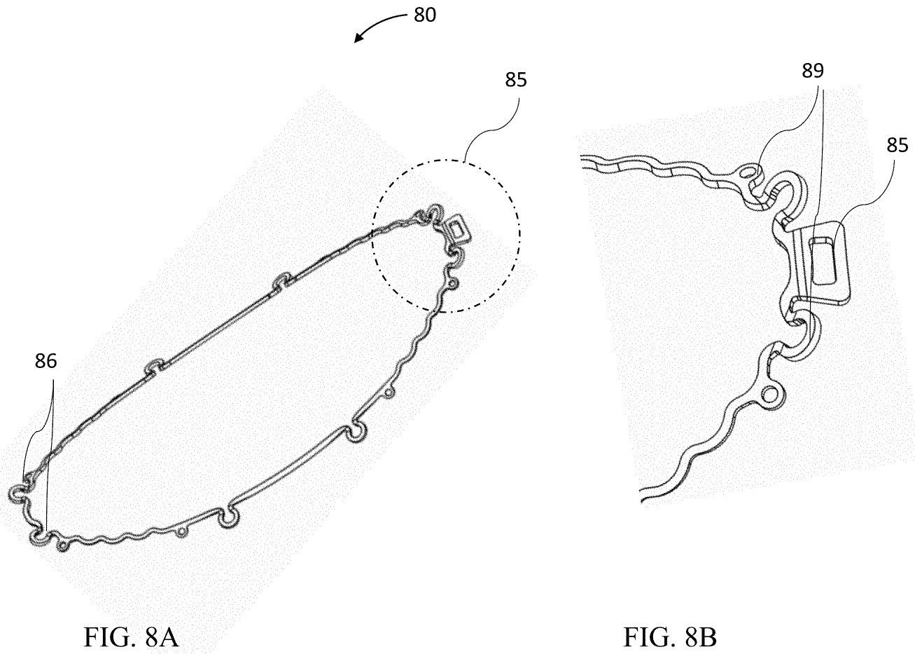

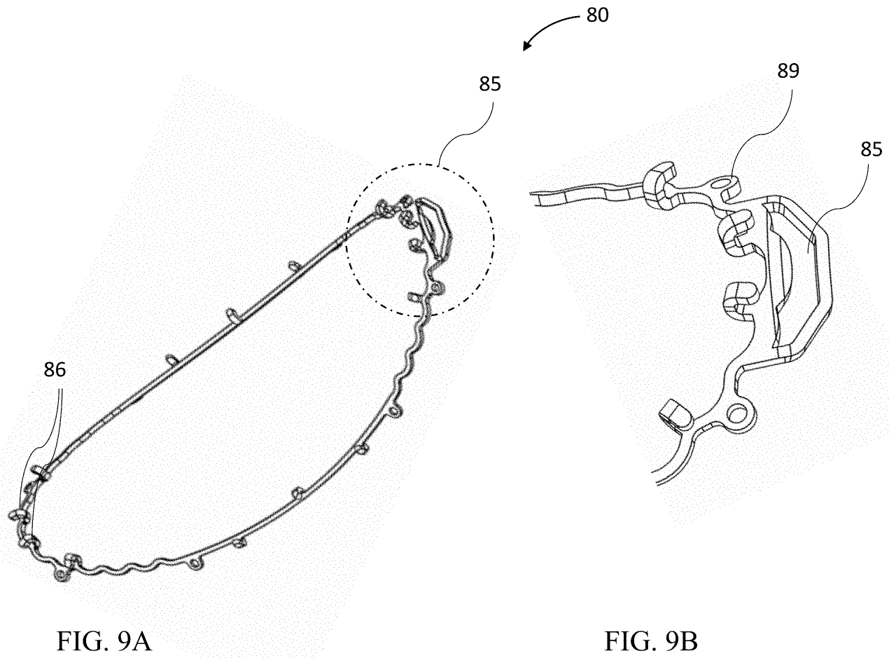

FIGS. 7-9B illustrate various configurations of retaining means which may be used in conjunction with filter insert 70.

In some embodiments, insert frame 80 comprises distal retaining means, such as retaining means 85. In some embodiments, distal retaining means 85 can be a hook, a loop, a tine, a strut, a clip, a latch, or any combination thereof. In some embodiments, the distal retaining means 85 are used for engaging a proximal end of hoop or ring defining aperture 17, and/or corresponding features therein, such as features 87 in FIGS. 6A-6D.

In some embodiments, insert frame 80 comprises proximal retaining means, such as retaining means 86. In some embodiments, proximal retaining means 86 can be a hook, a loop, a tine, a strut, a clip, a latch, or any combination thereof. In some embodiments, the proximal retaining means 86 are used for engaging a proximal end of hoop or ring defining aperture 17, and/or corresponding features therein, such as features 88 in FIGS. 6A-6D.

In some embodiments, insert frame 80 further comprises one or more side retaining means, such as retaining means 89. In some embodiments, side retaining means 89 can be a hook, a loop, a tine, a strut, a clip, a latch, or any combination thereof. In some embodiments, side retaining means 89 are used for engaging side portions of hoop or ring defining aperture 17, and/or corresponding features therein.

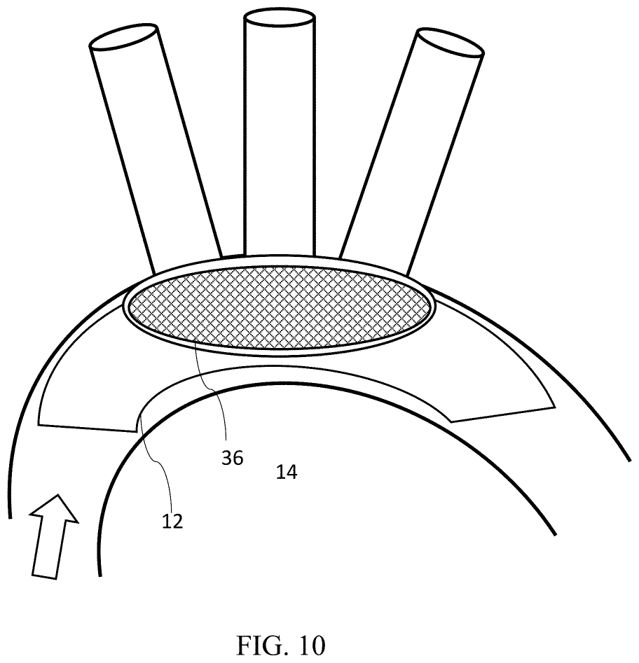

FIG. 10 shows a device of the present disclosure positioned in the aortic arch, while filter insert 70 covers the openings to the cerebral arteries.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” refers to at least 80%, at least 85%, at least 90%, at least 92%, at least 95%, at least 96%, at least 97%, at least 98%, at least 99%, including any range or value therebetween. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1.-34. (canceled)

35. A device comprising:
a tubular anchoring element configured and dimensioned for implanting about a multifurcation zone of a body lumen dividing a main vessel into at least two branches, to anchor said device therein,
wherein said anchoring element comprises an aperture about an outer wall region thereof encompassing at least inlets of said at least two branches, and wherein said aperture is defined by a perimeter ring;
a removable insert comprising a resiliently bendable frame having a periphery, and a filter unit within said periphery, said filter unit having a mesh size sufficient to allow passage of blood and to deflect the flow of embolic material exceeding a predetermined size,
wherein said removable insert is dimensioned for being received within said ring, and detachably secured thereto using one or more retaining means configured for engaging corresponding features in said ring.

36. The device of claim 35, wherein said anchoring element is formed of a self-expandable braided framework able to expand from a radially compressed state in a delivery configuration to a radially expanded state.

37. The device of claim 35, wherein said anchoring member comprises two or more circumferential strut sections disposed at spaced-apart axial positions along a longitudinal dimension thereof.

38. The device of claim 37, wherein each of said two or more circumferential strut sections is a radially expandable stent section configured to engage radially the walls of said body lumen.

39. The device of claim 37, wherein said two or more circumferential strut members are interconnected.

40. The device of claim 35, wherein said anchoring member comprises a fabric sheet attached to and covering circumferentially at least a longitudinal portion of said anchoring element.

41. The device of claim 35, wherein said removable insert is configured for in-situ (i) deployment within said aperture, and (ii) removal from said aperture.

42. The device of claim 35, wherein said removable insert is dimensioned for being freely movable within a lumen of said anchoring element.

43. The device of claim 35, wherein said periphery has a shape selected from the group consisting of: circular, oval, and rectangular.

44. The device of claim 35, wherein said removable insert is resiliently bendable along at least one of: a longitudinal dimension and a transverse dimension.

45. The device of claim 35, wherein said removable insert is substantially planar.

46. The device of claim 35, wherein said removable insert is pre-shaped with a radius of curvature along at least one of: a longitudinal dimension and a transverse dimension.

47. The device of claim 35, wherein said removable insert comprises one or more struts traversing said frame.

48. The device of claim 35, wherein said removable insert comprises said retaining means at least at a distal end and a proximal end thereof.

49. A method comprising:
implanting a tubular anchoring element about a multifurcation zone of a body lumen dividing a main vessel into at least two branches,
wherein said anchoring element comprises an aperture about an outer wall region thereof encompassing at least inlets of said at least two branches, and wherein said aperture is defined by a perimeter ring;
delivering into a lumen of said anchoring element, a removable insert comprising a resiliently bendable frame having a periphery, and a filter unit within said periphery, said filter unit having a mesh size sufficient to allow passage of blood and to deflect the flow of embolic material exceeding a predetermined size,
wherein said removable insert is dimensioned for being received within said ring, and detachably secured thereto using distal and proximal retaining means configured for engaging corresponding distal and proximal features in said ring;
engaging said distal corresponding features using said distal retaining means;
advancing said removable insert distally, so as to cause said removable insert to resiliently bend at least along a longitudinal dimension thereof;
aligning said proximal retaining means with said proximal corresponding features; and
allowing said removable insert to allowed to resiliently return to its original shape, thereby engaging said proximal corresponding features using said proximal retaining means.

50. The method of claim 49, wherein said anchoring element is formed of a self-expandable braided framework able to expand from a radially compressed state in a delivery configuration to a radially expanded state.

51. The method of claim 49, wherein said anchoring member comprises two or more circumferential strut sections disposed at spaced-apart axial positions along a longitudinal dimension thereof.

52. The method of claim 51, wherein each of said two or more circumferential strut sections is a radially expandable stent section configured to engage radially the walls of said body lumen.

53. The method of claim 51, wherein said two or more circumferential strut members are interconnected.

54. The method of claim 49, wherein said anchoring member comprises a fabric sheet attached to and covering circumferentially at least a longitudinal portion of said anchoring element.

55.-61. (canceled)
